SEQUENCE LISTING (7 sequences)

SEQ ID NO: 1 - DNA, 759 bp, Lactobacillus brevis
  CDS (1)..(759)
atgtctaaccgtttggatggtaaggtagcaatcattacaggtggtacgttgggtatcggtttagctatcgccacgaagttcgttgaagaaggggctaaggtcatgattaccgaccggcacagcgatgttggtgaaaaagcagctaagagtgtcggcactcctgatcagattcaatttttccaacatgattcttccgatgaagacggctggacgaaattattcgatgcaacggaaaaagcctttggcccagtttctacattagttaataacgctgggatcgcggttaacaagagtgtcgaagaaaccacgactgctgaatggcgtaaattattagccgtcaaccttgatggtgtcttcttcggtacccgattagggattcaacggatgaagaacaaaggcttaggggcttccatcatcaacatgtcttcgatcgaaggctttgtgggtgatcctagcttaggggcttacaacgcatctaaaggggccgtacggattatgtccaagtcagctgccttagattgtgccctaaaggactacgatgttcgggtaaacactgttcaccctggctacatcaagacaccattggttgatgacctaccaggggccgaagaagcgatgtcacaacggaccaagacgccaatgggccatatcggtgaacctaacgatattgcctacatctgtgtttacttggcttctaacgaatctaaatttgcaacgggttctgaattcgtagttgacggtggctacactgctcaatag

SEQ ID NO: 2 - protein, 252 aa, Lactobacillus brevis
MSNRLDGKVAIITGGTLGIGLAIATKFVEEGAKVMITDRHSDVGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINMSSIEGFVGDPSLGAYNASKGAVRIMSKSAALDCALKDYDVRVNTVHPGYIKTPLVDDLPGAEEAMSQRTKTPMGHIGEPNDIAYICVYLASNESKFATGSEFVVDGGYTAQ

SEQ ID NO: 3 - DNA, 24 bp, Artificial Sequence
  synthetic DNA
accgaccggcacagcgatgttggt

SEQ ID NO: 4 - protein, 8 aa, Artificial Sequence
  synthetic peptide
TDRHSDVG

SEQ ID NO: 5 - DNA, 24 bp, Artificial Sequence
  synthetic DNA
accaacatcgctgtgccggtcggt

SEQ ID NO: 6 - protein, 8 aa, Artificial Sequence
  synthetic peptide
GVDSHRDT

SEQ ID NO: 7 - protein, 8 aa, Artificial Sequence
  synthetic peptide
TDRHSDVG

(19) United States
(12) Patent Application Publication
(10) Pub. No.: US 2003/0054520 A1
(43) Pub. Date: Mar. 20, 2003

(54) Recombinant enzymes having improved NAD (H) affinity

(75) Inventors: Bettina Bommanus, Atlanta, GA (US); Werner Hummel, Titz (DE); Andreas Bommarius, Atlanta, GA (US)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: DEGUSSA AG, Duesseldorf (DE)

(21) Appl. No.: 09/910,033

(22) Filed: Jul. 23, 2001

(30) Foreign Application Priority Data
Jul. 27, 2000 (DE) 100 37 101.9

(51) Int. Cl. C12N 9/04; C12P 21/02; C12N 1/21; C12N 15/74

(52) U.S. Cl. 435/190; 435/69.1; 435/455; 435/320.1; 435/252.3

(57) ABSTRACT

The present invention relates to recombinantly modified enzymes, which exhibit increased NAD(H) affinity compared to a unmodified or wildtype enzyme, gene sequences or polynucleotides that code for the recombinantly modified enzymes, plasmids and microorganisms that contain these gene sequences; as well as, methods of making and methods of using the enzymes of the present invention.

### CROSS-REFERENCE TO RELATED APPLICATION

The present application claims priority to German Application DE 100 37101.9 filed Jul. 27, 2000, the entire contents of which are incorporated herein by reference.

### BACKGROUND OF THE INVENTION

1. Field of the Invention

The present invention relates to recombinantly (rec) modified enzymes. In particular, the invention relates to the recombinantly modified enzymes that exhibits increased NAD(H) affinity compared to a unmodified or wildtype enzyme. The invention also relates to gene sequences or polynucleotides that code for the recombinantly modified enzymes, plasmids and microorganisms that contain these gene sequences. The inventive enzymes may be employed to enatiomerically reduce or oxidize types of organic compounds.

### DISCUSSION OF THE BACKGROUND

The use of enzyme techniques in the synthesis of organic compounds is advantageous on the large industrial scale because such enzyme techniques are often superior to the normal chemical techniques as to selectivity and product yields.

In some cases, such enzyme techniques are dependent on so-called cofactors or coenzymes. For example, alcohol dehydrogenases (ADH) are enzymes which transform ketones to the corresponding alcohols with high enantioselectivity. The coenzyme in such reactions is very often NADH or NADPH. Most known ADHs (for example, from horse liver, or from the bacterium Thermoanaerobium brockii) form (S)-alcohols during use of comparable ketones. Nevertheless, two (R)-specific ADHs which are biochemically very similar are known from Lactobacillus strains, one being an enzyme from Lactobacillus kefir (European Patent 91107067.0; German Patent 4014573) and the other from L. brevis (European Patent 0796914 A2; German Patent 19610984; DSM 20054). A restriction in the use of these two R-specific enzymes exists due to the dependence on the coenzyme NADP(H). This coenzyme is considerably more unstable and more expensive than the coenzyme NADP(H), for which an established and cost-effective regeneration method does not exist. Because of the abnormally broad acceptance for ketones, which are transformed with almost complete enantiomeric purity by these enzymes, they are nevertheless of great interest for preparative applications.

In previous attempts to shift the coenzyme specificity of NADP(H) toward NAD(H), what has taken place heretofore has been predominantly “multiple” replacements of relatively large regions, which do not allow any systematic procedure to be discerned and which cannot be adopted for other NADP(H)-dependent enzymes (Chen, R. et al. (1995), “A highly active decarboxylating dehydrogenase with rationally inverted coenzyme specificity”, Proc. Natl. Acad. Sci. USA 92(25): 1166670; Perham, R. N. et al. (1991), “New enzymes for old: redesigning the coenzyme and substrate specificities of glutathione reductase”, Bioassays 13(10)): 515-25; Yaoi, T. et al. (1996), “Conversion of the coenzyme specificity of isocitrate dehydrogenase by module replacement”, J. Biochem. (Tokyo) 119(5): 1014-8). Only one publication (Sem, D. S. and C. B. Kasper (1993), “Interaction with arginine 597 of NADPH-cytochrome P-450 oxidoreductase is a primary source of the uniform binding energy used to discriminate between NADPH and NADH”, Biochemistry 32(43): 11548-58) describes a singular replacement on a dehydrogenase (cytochrome P450 oxidoreductase), although this was achieved in a manner analogous to that of WO99/47648. The authors replaced a basic amino acid by a neutral amino acid (Arg59/Met). The results of the authors confirm a slight improvement of NAD affinity, but the enzyme obtained is clearly more unstable.

Other attempts have been made employing genetic engineering methods to change the enzyme from L. brevis so that it can accept not only NADP(H) but also NAD(H) (WO99/47648). To achieve this change in coenzyme affinity, basic amino acids were substantially replaced by neutral amino acids at the coenzyme binding site. This replacement was achieved by changing the nucleotide sequence coding for the (R)-ADH from L. brevis. Thus the basic amino acids arginine-38, lysine-45 and lysine-48 were replaced in various combinations by neutral amino acids (such as methionine, leucine, isoleucine, glycine) in the region of the coenzyme binding site (the amino acid positions enumerated here include the start codon ATG). While these enzyme mutants were found to accept NAD(H) they proved to have little value for practical application, because the enzyme yields were relatively low and, in particular, the stabilities of these new enzymes are considerably poorer than the NADP(H)-dependent wild-type enzymes. Other mutants in which an additional replacement of a neutral amino acid by an acidic amino acid (G38D) was performed along with the above-mentioned replacements of basic amino acids by neutral amino acids (replacements R39L, K48M as well as the charge neutral replacement A9G), indeed exhibited broadening of the coenzyme affinity toward NAD(H), but was also considerably unstable and obtainable only with low yields.

However, there remains a critical need for enzymes with improved NAD(H) affinity and thus for methods of producing such enzymes. These enzymes can be employed to transform ketones to their corresponding alcohols with high enantioselectivity. On a commercial or industrial scale even small improvements in these conversions, or the efficiency of their production, are economically significant. Prior to the present invention, it was not recognized that by modifying an enzyme to replace at least one acidic amino acid with at least one neutral amino acid while retaining the basic amino acids at the coenzyme binding site of the enzyme would improve NAD(H) affinity of the enzyme, maintain excellent stability and thus provide for enzymes that can be effectively used for, e.g., ketone/alcohol conversions. The previously existing natural preference for the unstable coenzyme NADP(H) can therefore be shifted toward the preferred and advantageous NAD(H) affinity by the replacement of only one amino acid. This cannot be inferred as such from the prior art, and is therefore very surprising. In experiments, it has been found that the affinity for NAD(H) compared with NADP(H) in the inventive modified enzyme can be increased by a factor of about 300 by this replacement, without impairing the stability of the rec-enzyme.

To the contrary, the thermal stability increases.

The object of the present invention was therefore to specify a general method and enzymes obtained by means of this method which makes it possible to increase the inherently unnatural NAD(H) affinity of the enzymes without at least substantially impairing their stability.

### SUMMARY OF THE INVENTION

Accordingly, one object of the present invention is a modified enzyme wherein at least one acidic amino acid is replaced with at least one neutral amino acid and wherein the basic amino acids at the coenzyme binding site of said enzyme are not replaced; wherein the modified enzyme exhibits increased NAD(H) affinity compared to an unmodified enzyme.

In one aspect of the invention, the enzyme is a dehydrogenase enzyme, an alcohol dehydrogenase enzyme, a rec-(R)-alcohol dehydrogenase enzyme, and a L. brevis or L. kefir rec-(R)-alcohol dehydrogenase enzyme.

In another aspect of the invention the enzyme has the amino acid sequence of SEQ ID NO:2.

Another aspect of the present invention are isolated genes or polynucleotides which encodes the modified enzyme. One example of such a polynucleotide is the nucleotide sequence of SEQ ID NO: 1.

Another aspect of the present invention are plasmid vectors containing the isolated polynucleotides or genes that encode the enzyme and as to host cells, e.g., microrganisms such as bacteria or yeast.

Another object of the present invention is to provide methods of modifying an enzyme comprising: replacing at least one neutral amino acid in said enzyme with at least one acidic amino acid, wherein the basic amino acids at the coenzyme binding site of said enzyme are not replaced; and wherein said modified enzyme exhibits increased NAD(H) affinity compared to an unmodified enzyme.

Another object of the present invention is to provide a method for producing the enzymes of the invention for a time and under conditions suitable for the expression of the polynucleotide which encodes said enzyme; and collecting the enzyme.

Another object of the present invention is to provide methods for the enantioselective reduction of a organic compound comprising reacting the compound with the enzyme and at least one of NAD(H) and NAD+, wherein said organic compound is selected from the group selected from the group consisting of ketones, α-keto esters, β-keto esters, γ-keto esters, and combinations thereof.

Another object of the present invention is to provide methods for the enantioselective oxidation of alcohols comprising reacting an alcohol comprising reacting a alcohol with the enzyme and at least one of NAD(H) and NAD+.

### BRIEF DESCRIPTION OF THE DRAWINGS

FIG. 1: SDS-PAGE purification of the G38D mutein.

FIG. 2: pH optimum of the G38D mutein.

FIG. 3: pH stability of the G38D mutein.

FIG. 4: Thermal stability of the G38D mutein at 50° C.

FIG. 5: Thermal stability of the G38D mutein at 30° C.

FIG. 6: Temperature optimum of the G38D mutein

FIG. 7: Map of the pBTAC2 vector

### DETAILED DESCRIPTION OF THE INVENTION

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety.

Reference is made to standard textbooks of molecular biology that contain definitions and methods and means for carrying out basic techniques, encompassed by the present invention. See, for example, Current Protocols in Molecular Biology, Ausbel et al (eds.), (2000 edition), John Wiley and Sons, Inc. NY; Sambrook et al., Molecular Cloning: A Laboratory Manual, Cold Spring Harbor Laboratory Press, Cold Spring Harbor, New York (1989) and the various references cited therein.

By means of the inventive method, all NADP(H)-dependent enzymes known to the person skilled in the art can in principle be appropriately modified. Preferably such an enzyme is a dehydrogenase, especially an alcohol dehydrogenase. Even more especially, however, this is achieved for the (R)-ADH from L. brevis or L. kefir. In this way, rec-(R)-ADHs with the advantages cited hereinabove are advantageously obtained from the said organisms. Most especially preferred are such rec-(R)-ADHs from L. brevis or L. kefir in which a G was replaced by a D as the amino acid at position 38. As regards the position identification, the start amino acid corresponding to the codon ATG is included in the count.

A further embodiment of the invention relates to gene sequences which code for the inventive rec-enzyme. Subject matter of the invention is also plasmids and microorganisms containing the inventive gene sequences. The microorganism in which the gene sequence is cloned is used for multiplication and production of an adequate quantity of the recombinant enzyme. The methods for this purpose are well known to the person skilled in the art (Sambrook et al., 1989, Molecular Cloning: A Laboratory Manual, 2nd Edition, Cold Spring Harbor Laboratory Press; Balbas P & Bolivar F., 1990, Design and construction of expression plasmid vectors in E. coli, Methods Enzymology 185, 14-37). In principle, all organisms known for this purpose by the person skilled in the art can be used as the microorganisms. Preferably E. coli strains will be used for this purpose. Especially preferred are: E. coli NM 522, JM105, RR1, DHSa, TOP 10- or HB101. Plasmids with which the gene construct containing the inventive gene sequence is preferably cloned in the host organism are: pKK-177-3H (Roche Biochemicals), pBTac (Roche Biochemicals), pKK-233 (Stratagene) or pET (Novagen). Further options are also known in principle by the person skilled in the art (see the literature cited hereinabove, or at any rate the corresponding specialized molecular biology catalogs).

The enzymes of the present invention can be prepared by culturing the host cells, preferably such host cells are bacterial or yeast host cells, in a culture medium for a time and under conditions suitable for the expression of the polynucleotide or gene which encodes the recombinant or modified enzyme followed by collecting the enzyme from the host cell culture after it has been expressed. Methods of culturing cells lines to yield expression of the enzyme and thus obtaining the enzyme are known to the skilled artisan, as well as methods by which the enzyme can be recovered or purified from the host cell culture. Examples of such protocols are described in Current Protocols in Molecular Biology, Ausbel et al (eds.), (2000 edition), John Wiley and Sons, Inc. NY; Sambrook et al., Molecular Cloning: A Laboratory Manual, Cold Spring Harbor Laboratory Press, Cold Spring Harbor, New York (1989) and the various references cited therein.

The primer strands necessary for the PCR form a further part of the present invention. The sense and antisense primers coding for the amino acid sequence TDRHSDVG are also included.

Another aspect of the invention relates to a method for preparation of recombinantly prepared enzymes with NAD(H) affinity increased compared with the wild type. This is achieved by the fact that at least one neutral amino acid is replaced by at least one acidic amino acid, while retaining the basic amino acids at the coenzyme binding site of the enzyme. Generally, the inventive method for modifying the enzyme requires advance knowledge or preliminary determination of the amino acid sequence of the enzyme to be improved, in order to be able to achieve selective replacement of the corresponding amino acids. The replacement that is effective for improvement of NAD(H) specificity is also ascertained, however, by the trial-and-error principle—without prior knowledge of the coenzyme binding site—for which mutagenesis protocols and ready-to-use mutagenesis kits are now commercially available make it possible to perform the most important subordinate steps of the genetic engineering studies with little time and effort (see, for example, Maniatis et al., Molecular Cloning: A Laboratory Manual, Cold Spring Harbor Laboratory, New York (1982) and Sambrook et al., Molecular Cloning: A Laboratory Manual, Cold Spring Harbor Laboratory, New York (1989); and the specialized catalogs of Qiagen or Clontech).

The inventive method is preferably applied to a dehydrogenase, especially an alcohol dehydrogenase, and more preferably to the rec-(R)-ADH from L. brevis or L. kefir. Recombinant mutants (rec-mutants) of the (R)-ADHs (muteins) are advantageously obtained from these organisms. Most preferred are such rec-(R)-ADHs from L. brevis or L. kefir in which a G is replaced by a D as the amino acid at position 38. The position identification or count relates to the start amino acid corresponding to the ATG codon.

Examples of other enzymes that can be employed in the present invention include 2,5-Diketogluconic acid reductase whose product is 2-keto-1-gulonate (a sugar), which is often employed as a precursor of vitamin C production (Salini G. and M. Blaber (2001), Structural assembly of the active site in an aldo-keto reductase by NADP(H) cofactor. J Mol Biol 309:1209-1218); Dehydroascorbate reductase whose product is Ascorbic acid (a vitamin), which is often employed in pharmaceuticals or foods (Del. Bello et al (1994) Purification of NADPH-dependent dehydroascorbate reductase. Biochem J 304:385-390); 1,5-Anhydro-D-fructose reductase whose product is 1,5-anhydro-D-glucitol (a sugar), which is often employed as a building block (Sakuma et al (1998) Purification and some properties of a hepatic NADPH-dependent reductase that specifically acts on 1,5-anhdro-D-fructose. J Biochem (Tokyo) 123:189-193); Dihydrofolate reductase whose product is (6S)-tetrahydrofolate (a vitamin), which often employed as a precursor of leucovorin (Eguchi et al (1992) NADPH regeneration by glucose dehydrogenase from Gluconobacter scleroides for 1-leucovorin synthesis. Biosci Biotechnol Biochem 56:701-703); Carbonyl reductase (Candida magnoliae) whose product is Ethyl-(S)4-chloro-3-hydroxy-butanoate (hydroxy acid, which is often employed as a building block (Wada et al (1998) Purification and characterization of NAPH-dependent carbonyl reductase, involved in stereoselective reduction of ethyl 4-chloro-3-oxobutanoate, from Candida Magnoliae. Biosci Biotechnol Biochem 62:280-285); Glutamate dehydrogenase whose product is L-glutamate (amino acid), which is often employed in foodstuffs (Srinivasan R. (1991) Characterization of the general anion-binding site in glutamate dehydrogenase-NADPH complex. Biochim Biophys Acta 1073:18-22); Tylosin reductase whose product is Relomycin (antibiotic), which is often employed in pharmaceuticals (Huang et al (1993) Purification and properties of NADPH-dependent tylosin reductase from Streptomyces fradiae. J Biol Chem 268:18987-18993); Carbonyl reductase (Candida macedoniensis) whose products are Chiral alcohols and polyalcohols, which are often employed as building blocks (Kataoka et al (1992) A novel NADPH-dependent carbonyl reductase of Candida macedoniensis: purification and characterization. Arch Biochem Biophys 294:469-474); Alcohol dehydrogenase (Thermoanaerobium brockii) whose products are Chiral (S)-alcohols, which are often employed as building blocks (Keinan et al (1987) Synthetic applications of alchol-dehydrogenase from Thermoanaerobium brockii. Ann. N.Y. Acad. Sci. 501:130-149); and other related enzymes.

Another aspect of the invention is related to the use of an inventive rec-enzyme in a method for preparing enantiomerically enriched organic compounds, preferably enantiomerically enriched alcohols. Preferably the rec-(R)-ADH from L. brevis or L. kefir is used in the method for enantioselective reduction of ketones or for enantioselective oxidation of alcohols.

The inventive rec-enzymes may be prepared by genetic engineering methods known to the person skilled in the art (for example, Sambrook et al., 1989, loc cit.; Vectors: A Survey of Molecular Cloning Vectors and Their Uses, R. L. Rodriguez & D. T. Denhardt, Eds.: 205-225). Generally, methods such as PCR, fusion PCR, cloning, and expression may be employed, see WO99/47684 and the references cited therein. The positive change of the mutated rec-enzymes can be demonstrated by determining the kinetic parameters for coenzymes NAD+, NADP+, NADH and NADPH with the corresponding kinetic parameters for the ketone substrate.

Preferably, a modified enzyme prepared as described above will have a Km that is at least 2 times, more preferably 5, 10, 15, 20, 25, 50, 100, 150, 200, or 300 times lower than the unmodified enzyme. The lower Km thereby results in a increased affinity to NAD(H). Accordingly, the terms “increased”, “improved” and “enhanced” as used herein are understood to mean those modified enzymes which have the amino acid alterations described herein and that have the lower Km or higher affinity to NAD(H) as compared to an unmodified or wildtype enzyme as described herein.

From biochemical comparison of those mutants produced according to International Patent Application WO99/47684 with the inventive rec-(R)-ADH described herein, the following advantageous improvements are apparent in the G38D mutant.

The mutant has considerably better thermal stability, this enzyme being much more stable than the non-mutated NADP(H) converting wild-type enzyme.

The Km for NADP is higher and thus the affinity for NADP is poorer compared with the wild-type enzyme. In contrast, the Km for NAD has become lower and thus the affinity has been improved. The plasmid stability that contains the G38D mutant gene is much better compared to plasmids with genes produced according to WO99/47684.

The gene expression yield of the G38D replacement (+ATG start codon) is much higher than that of the enzymes produced according to WO99/47684.

These new properties of the inventive rec-(R)-ADH achieved by a single replacement lead to an enzyme which is highly suitable for preparative applications. It accepts the more cost-effective and more stable NAD(H) instead of NADP(H), has high stability and exhibits advantageous biochemical properties. It can be used both for reductions of ketones to chiral alcohols (equation (1) below) and for oxidation reactions (equation (2) below). In addition to ketones, keto esters (such as α-, β-, γ-keto esters) are accepted very effectively.

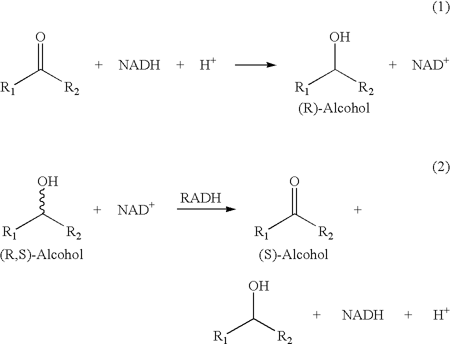

For preparative applications according to equation (1), the option of using NADH is particularly advantageous, because known methods (formate/formate dehydrogenase) for the necessary regeneration of NADH may be employed. Since the binding site for ketones or alcohols has not been changed by the mutation, the known broad range of application of the rec-(R)-ADH can be fully exploited using NAD(H).

Gene sequences which code for amino acid sequences include all sequences which the skilled artisan will recognize as possible based on the degeneracy of the genetic code.

In the scope of the invention, enantiomerically enriched means the fact that, in the mixture of two optical antipodes, one is present in a proportion of greater than 50%.

### EXAMPLES

### Example 1

### Description of Preparation of the Mutant recADHG38D:

The template used for preparation of this mutant was the gene of the wild-type enzyme present as a clone in E. coli.

Starting from the primary sequence of the wild-type enzyme, and taking into consideration the knowledge of the spatial structure of this wild-type ADH, genetic primers were defined and used in such a way that a replacement of glycine by aspartic acid was performed at position 38 with the “polymerase chain reaction” method (PCR).

Primers for the directed mutagenesis of the change of cofactor specificity from NADP to NAD (the desired amino acid replacement is indicated in bold italics):

5′-Primer with the G38Ds amino acid replacement:

| | |
|---|---|
| 5′ACC GAC CGG CAC AGC GAT GTT GGT 3′ | (SEQ ID NO 3) |
| T D R H S D V G | (SEQ ID NO:4) |

3′-Primer with the G38Das amino acid replacement:

| | |
|---|---|
| 5′-ACC AAC ATC GCT GTG CCG GTC GGT 3′ | (SEQ ID NO:5) |
| G V D S H R D T | (SEQ ID NO:6) |

In order to perform a mutation successfully, the nucleotide replacement responsible for the amino acid replacement must take place on both DNA strands, both on the leading strand (s=sense) and on the lagging strand (as=antisense). For the mutation PCR this means that 2 gene fragments are generated, one from the 5′-end of the gene up to the point in the gene where the amino acid replacement, and one from the point in the gene where the amino acid replacement to the 3′-end of the gene. These two gene segments then have an overlapping region in the aforesaid primer containing the amino acid replacement, or in other words the two gene fragments have in common the aforesaid amino acids of TDRHSDVG (SEQ ID NO:7). Via this common region, the two gene fragments can then be fused in a second PCR, known as fusion PCR.

PCR with the new mutation-specific primers for preparation of the short and long fragments:

| PCR | Template | 5′ Primer | 3′ Primer | dNTP | Buffer | DNAzyme | H₂O | Temp. |
|---|---|---|---|---|---|---|---|---|
| 1 | recADH | G38Ds | Bras | 16 μl | 10 μl | 0.5 μl | 69.5 μl | 56° C. |
| | WT 2 μl | 100 pmol | 100 pmol | | | | | |
| 2 | recADH | BRs | G38Das | 16 μl | 10 μl | 0.5 μl | 69.5 μl | 56° C. |
| | WT 2 μl | 100 pmol | 100 pmol | | | | | |

The gene fragments produced by this PCR are joined in the fusion PCR. For this purpose equal pmol ends of template from PCR 1 and PCR 2 were pipetted together and otherwise the ingredients as above were used, except for the primers.

The first 5 cycles of the PCR were performed without any primer, and after the 5th cycle 100 pmol of BRs (N-terminus of the gene) and BRas (C-terminus of the gene) were added and a further 25 cycles performed. By virtue of the first 5 cycles without primer, it was ensured that only fused gene fragments can function as the template for the polymerase. Amplification then began after 5 cycles, with addition of the gene-specific primer.

In this way, genes with point mutations can be generated on both DNA strands.

| Fusion | | | | | | | | |
|---|---|---|---|---|---|---|---|---|
| PCR | Template | 5′ Primer | 3′ Primer | dNTP | Buffer | DNAzyme | H₂O | Temp. |
| 3 | PCR 1 | BRs | BRAS | 16 μl | 10 μl | 0.5 μl | 59.5 μl | 52° C. |
| | 1 pmol + | 100 pmol | 100 pmol | | | | | |
| | PCR 2 | | | | | | | |
| | 1 pmol | | | | | | | |

The fusion product (=G38D mutein of recADH) was isolated from the gel (Gel Extraction Kit, Qiagen) and purified. The gene was then cut corresponding to its joined 5′ and 3′ restriction cut points (Eco R1 and HindIII) and again isolated by gel electrophoresis and purified (see, for example, Patent WO99/47684).

The commercial vector pBTAC2 used here (Roche Diagnostics; formerly Boehringer Mannheim, see FIG. 7) was also restricted with EcoR1 and HindIII, and thus was prepared for cloning with the vector.

Cloning in the Vector pBTac2:

The restricted mutein was ligated into the vector pBTAC2 by means of the Rapid Ligation Kit (Roche Diagnostics) and then transformed in competent E. coli JM105 cells (60 sec, 42° C. heatshock) (or alternately also in E. coli SG13009 cells (Qiagen), which contain additional repressor plasmids with neomycin resistance, plasmid pREP4, commercially available from Qiagen).

The successfully transformed clones were tested as to their expression capability.

Expression of the G38D Mutein:

The mutein was induced with 1 mM IPTG at OD 0.5 in shaking flasks (LB medium) and the cells were harvested after 24 hours of expression. Ampicillin was used for selection pressure.

The G38D mutein was formed with very good expression capability, comparable with the expression of the wild-type enzyme. In the raw extract of the recombinant cells, about 30 to 40% of the total protein was formed as recombinant ADH G38D mutein, and the volume activity (tested with acetophenone/NADH) was 23 U/ml.

### Example 2

### Purification and Biochemical Characterization of the Mutant recADHG38D

The mutein was purified to almost homogeneous protein and characterized.

Purification of G38D Mutein of recADH:

The E. coli strain containing the mutein was digested with 0.1 M Na acetate of pH 4.5 (glass-beads digestion, IMA disintegrator S, 4000 rpm, 20 minutes, 4° C.) and the cell slurry was then centrifuged at 13000 rpm (Sorvall SS34 rotor, 4° C., 10 minutes). The cell-free supernatant contains the enzyme (raw extract). This raw extract was adjusted to 0.6 M with (NH₄)₂SO₄ and applied on a phenylsepharose column (25 ml SV, Pharmacia) equilibrated with 50 mM TEA of pH 7.0+0.6 M ammonium sulfate+1 MM MgCl₂. The protein was eluted with salt gradient decreasing to 0 M ammonium sulfate. The active fractions were united and concentrated by ultrafiltration (Amicon stirred cell). Ammonium sulfate up to 1.2 M was added to this active pool, whereupon the mixture was applied on an octylsepharose column equilibrated versus 50 mM TEA of pH 7.0+1 MM MgCl₂+1.2 M ammonium sulfate. The protein was eluted once again with a gradient decreasing to 0 M ammonium sulfate. The active eluate of this column was used for characterization studies.

Purification table

| | | | Specific | | | |
|---|---|---|---|---|---|---|
| | Activity | Protein | activity | | | |
| Sample | [U/ml] | [mg/ml] | [U/mg] | Yield [%] | Factor | Σ U |
| Raw extract | 23.2 | 5.78 | 4.02 | 100 | 1 | 511 |
| Phenyl- | 50 | 17.18 | 2.88 | 19 | 0.71 | 98 |
| sepharose | | | | | | |
| Octyl- | 8.36 | 1.22 | 6.85 | 4 | 1.7 | 20 |
| sepharose | | | | | | |

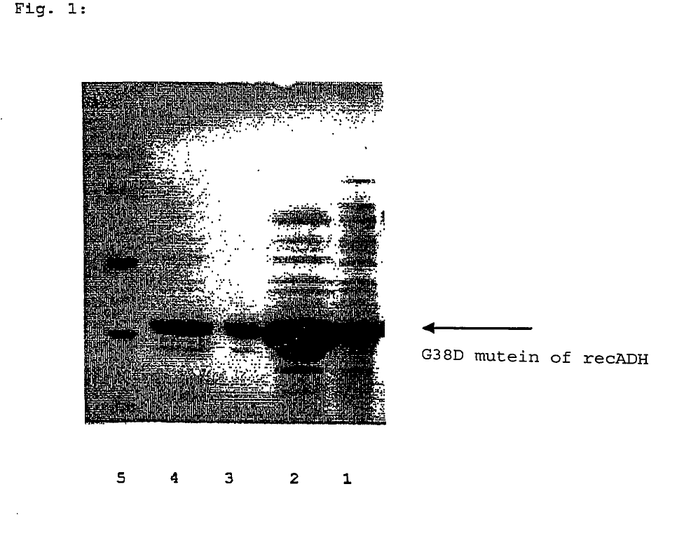

SDS PAGE of the G38D Mutein Purification (FIG. 1):

1 RE115 μg

2 PS 340 μg

3 Octyl1 31.6 μg

4 Octyl2 48.8 μg

As is evident from these data, the G38D mutein of the recADH is strongly overexpressed, and the smaller volume activity compared with the wild type is not due to lower expression capability. Lane 4 corresponds to the selected octyl pool in the above purification table.

Characterization of the G38D Mutein of recADH:

The mutein was characterized with respect to pH optimum, pH stability, temperature optimum, thermal stability, Km values for the oxidative direction, Kcat and Kcat/Km.

These criteria were compared with the wild-type enzyme and also with mutein 2 (mutant 2: recADH R39L,K49M,A1OG (counted with additional start codon), which is produced and described in Application WO99/47684.

pH Optimum of the G38D Mutein (FIG. 2):

The pH optimum of the mutein: the pH optimum of the reduction direction is 5.5, that of the oxidation direction is 6.5.

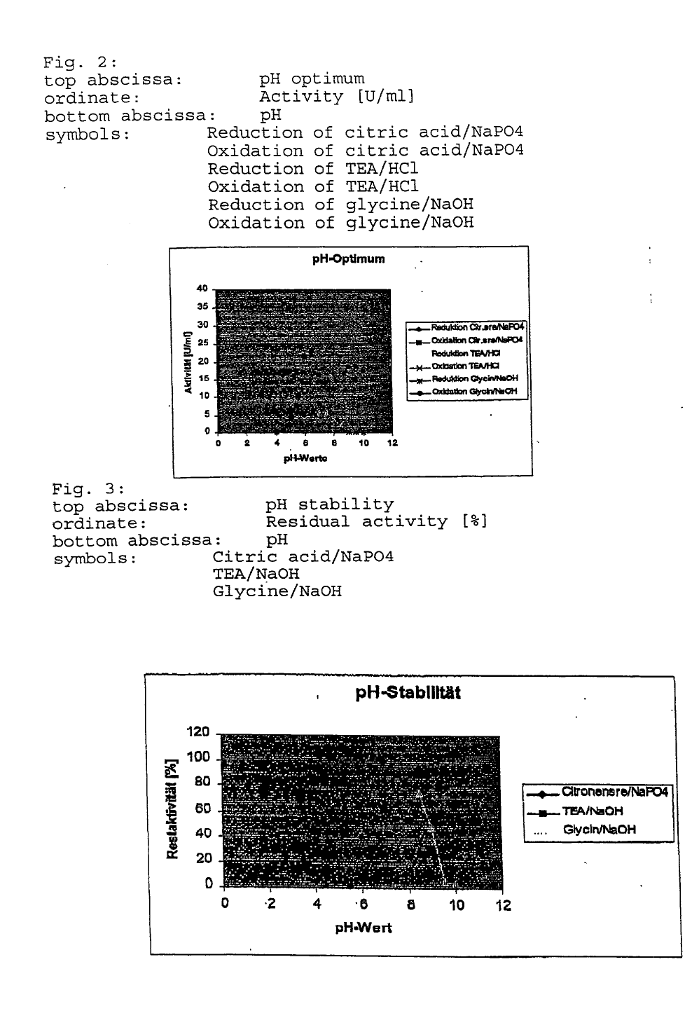

pH Stability of the G38D Mutein (FIG. 3):

The pH stability for each pH range was determined in different buffers; the enzyme is stable for at least 24 hours between 6.5 and 8.5, its being clearly evident that TEA buffer is not suitable for storage stability. For equal pH values the buffer always exhibits lower values than the others.

Thermal Stability (FIG. 4):

The thermal stability was determined with samples containing+/50% glycerol (final concentration). 50 μl of enzyme sample was covered with sufficient paraffin oil to prevent evaporation at high temperatures. Glycerol is absolutely necessary for prolonged stability of the enzyme, since otherwise it becomes denatured at temperatures or around 50° C. In comparison, the wild-type enzyme was measured with glycerol and mutant 2 (R39L K49M A1OG; WO99/47684) was measured without glycerol addition.

The thermal stability was measured at 42° C., the half-life of the mutein being 257 hours with glycerol addition.

| t, min | A, U/ml | in A | in A calc. | A calc., U/ml |
|---|---|---|---|---|
| 0 | 15 | 2.7080502 | 2.36981758 | 10.695441 |
| 1440 | 7.48 | 2.01223279 | 2.30501677 | 10.0243463 |
| 2880 | 8.12 | 2.09433015 | 2.24021596 | 9.39536008 |
| 4320 | 9.2 | 2.21920348 | 2.17541515 | 8.8058401 |
| 11520 | 6.74 | 1.90805992 | 1.8514111 | 6.36880021 |
| Slope | | −4.5001E-05 | | |
| Intercept on | 2.36981758 | | | |
| axis | | | | |

Table associated with FIG. 4.

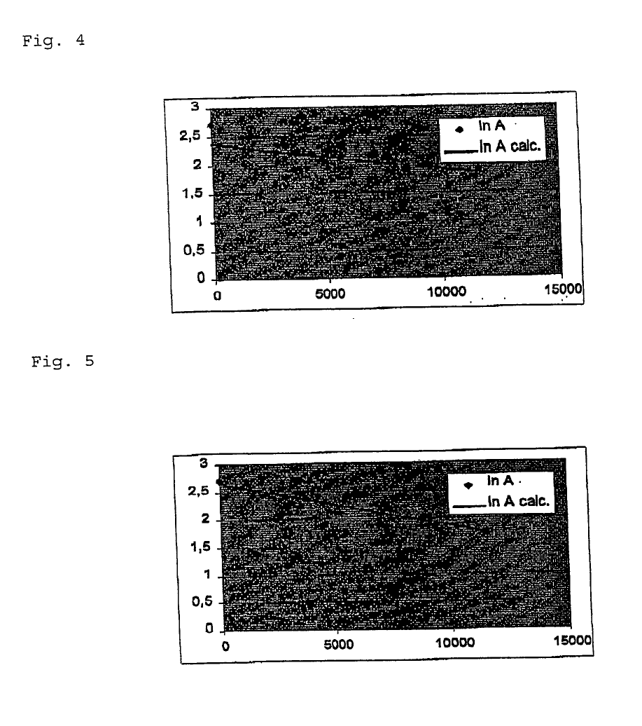

The thermal stability of the mutein was measured at 30° C., the half-life being 148 hours (FIG. 5)

| t, min | A, U/ml | in A | in A calc. | A calc., U/ml |
|---|---|---|---|---|
| 0 | 15 | 2.7080502 | 2.65518596 | 14.2276316 |
| 1440 | 11.32 | 2.42657107 | 2.54298444 | 12.7175693 |
| 2880 | 11.24 | 2.41947884 | 2.43078292 | 11.3677787 |
| 4320 | 11.14 | 2.41054223 | 2.3185814 | 10.1612493 |
| 11520 | 5.7 | 1.74046617 | 1.7575738 | 5.79835233 |

Table associated with FIG. 5.

Temperature Optimum:

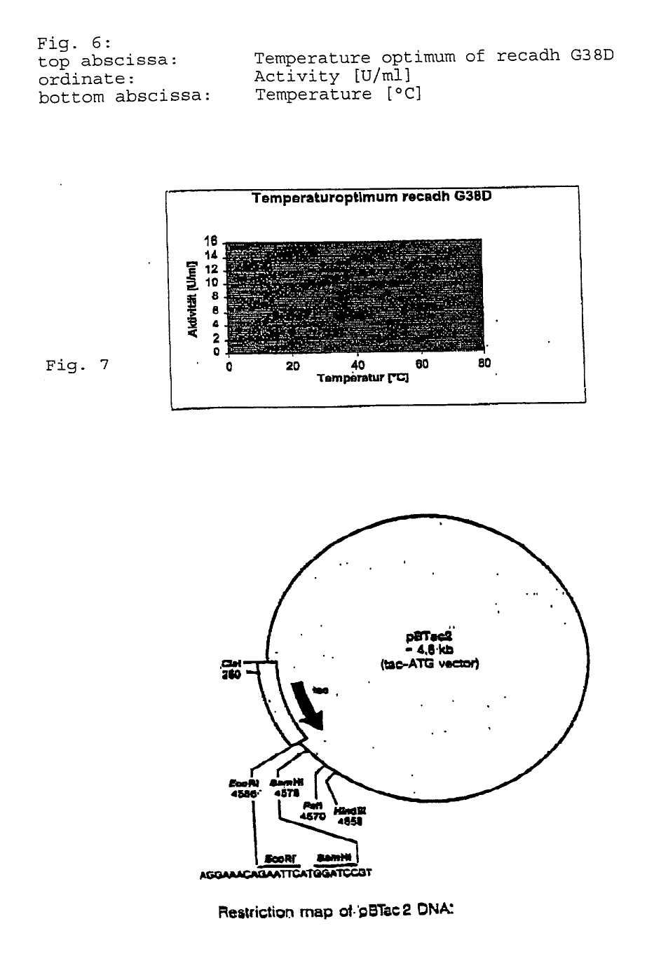

The temperature optimum was determined in the test batch in the vessel. The activity was measured with acetophenone and NADH (FIG. 6). The temperature optimum of the G38D mutein is 40° C.

### Example 3

### Comparison of the Biochemical Properties of the Mutant recADHG38D with a Mutant Prepared Per WO99/47684 and with the Wild-Type Enzyme

The Km values and all data related to Km or Vmax values are presented in the following overall table; the calculation of the values was performed by means of nonlinear regression with the program ORIGIN.

TABLE
Summary and comparison of all characteristics of the G38D mutein and
mutant 2 (W099/47684) with the wild-type enzyme

| | Wild-type | | |
|---|---|---|---|
| Characteristics | enzyme | Mutant 2 | G38D mutein |
| pH optimum for | 6.5 | 6.5 | 5.5 |
| reduction | | | |
| pH optimum for | 8.0 | 6.5 | 6.5 |
| oxidation | | | |
| pH for 24 hours | 4.5-9.0 (70%) | 5.5-8.5 (70%) | 6.5-8.5 (80%) |
| stability | | | |
| Temperature | 55 | 50 | 40 |
| optimum [° C.] | | | |
| Thermal stability at | 150 h* | 16.5 h | 148 h* |
| 30° C. | | | |
| Thermal stability at | 7.15 h* | 0.19 h | 257 h* |
| 42° C. | | | |
| Km NAD [mM] | 2.94 | 0.77 | 0.89 |
| Km NADP [mM] | 0.24 | 0.11 | 14.04 |
| Vmax NAD | 467 | 439 | 236 |
| [nMol/ml*s] | | | |
| Vmax NADP | 1420 | 623 | 402 |
| [nMol/ml*s] | | | |
| kcat NAD [s⁻¹] | 21.4 | 33.11 | 34.57 |
| kcat NADP [s⁻¹] | 65.2 | 46.98 | 58.88 |
| kcat/Km NAD | 7.3 | 43 | 38.84 |
| [s⁻¹*mM⁻¹] | | | |
| kcat/Km NADP | 270 | 427 | 4 |
| [s⁻¹*mM⁻¹] | | | |
| NAD:NADP*** | 0.03:1 | 0.1:1 | 10:1 |

*with 50% glycerol
***What was calculated was the ratio of kcat/Km for NAD to kcat/Km for NADP as a quantitative measure of the affinity of the two coenzymes.

The improvement of the G38D mutein lies in distinctly improved affinity of NAD. The summary table makes it clear that the wild-type enzyme can convert NAD only in a ratio of 0.03:1, whereas the new mutein described hereinabove accepts NAD 10 times better than NADP. Furthermore, the inventive mutein has distinctly improved thermal stability compared with the wild-type enzyme (both measured with glycerol in buffer), especially at higher temperatures (42° C.). The thermal stabilities are always presented as half-lives, where t1/2 denotes the time where the measured residual activity is still 50%. Good thermal stability is generally regarded as a measure of good long-term stability under production conditions.

### Example 4

### Substrate Spectrum of the Mutant recADHG38D

It is known that the NADP(H)-dependent wild-type enzyme can reduce numerous ketones, keto esters and other carbonyl-groupcontaining compounds stereospecifically. Hereinafter, only a few selected keto compounds are tested as substrates, in order to confirm that the substrate-recognition region has not been changed in principle by the change of coenzyme binding site. For this purpose the keto compounds are tested in the following mixture (total volume of 1 ml).

10 mM keto substrate; 1 MM MgCl₂.6H₂O; 0.4 mM NADH; 960 μl of triethanolamine buffer, 50 mM, pH 7.0; 10 μl of enzyme (G38D mutein); partly purified (phenylsepharose; see above).

The activity is determined photometrically at 340 nm (30° C.). The following table summarizes the activity values.

TABLE
Substrate spectrum of the NAD G38D mutant (activities expressed relative
to acetophenone (= 21.08 U/ml))

| | Activity, |
|---|---|
| Substrate | relative [%] |
| Acetophenone | 100 |
| 4-chloroacetophenone | 68 |
| 2-Hexanone | 169 |
| 2-Heptanone | 207 |
| 2-Methylcyclohexanone | 334 |
| Acetoacetic acid methyl ester | 188 |
| Acetoacetic acid ethyl ester | 88 |
| 4-Chloroacetoacetic acid ethyl ester | 228 |
| Pyruvic acid methyl ester | 191 |
| Pyruvic acid ethyl ester | 260 |
| 2-Oxobutyric acid ethyl ester | 137 |
| 3-Methyl-2-oxobutyric acid ethyl ester | 84 |
| Benzyl pyruvate ethyl ester | 13 |
| Phenylglyoxylic acid methyl ester | 10 |
| 3-Oxovaleric acid methyl ester | 127 |

### Example 5

### Demonstration of the Stereoselectivity of the G38D Mutein of recADH:

The enantiomeric purity of the product formed by reduction will be demonstrated for individual, selected keto substrates. For this purpose the substrates are converted largely completely, accompanied by coenzyme regeneration, and the enantiomeric purity of the product is determined by means of gas chromatography.

Conversion (1 ml total):

10 mM keto substrate; 1 MM MgCl₂.6H₂O; 1 mM NADH; 100 mM Na formate; 0.8 U of formate dehydrogenase; 2 U of NAD mutant (units determined photometrically with acetophenone/NADH); 680 μl of triethanolamine buffer, 50 mM, pH 7.0.

Samples (50 μl) are taken after 30 and 120 minutes respectively, 100μl of ethyl acetate is added for extraction of the product, and the ethyl acetate phase (1μl) is used for the GC analysis. Separation of the enantiomers by GC is checked for each product by application of the racemate. The purity of the product is expressed as the ee value, obtained as:

ee(R)=[R]−[S]/[R]+[S]

If S-enantiomer is not detectable, the ee value is given as >99%.

GC Analysis

Column: CP Chirasil DEX CB, length: 25 m, diameter: 25 μm (Chrompack Co.). Temperature program: 5 minutes at 60° C., then 5° C./minute up to 190° C. (for hexanone/hexanol: 30 minutes at 60° C., then 10° C./minute up to 195° C.). Column flowrate 1.3 Ml/minute; gas: helium.

The following table summarizes the data on product purity.

TABLE
Demonstration of enantiomeric purity of the products formed by enzyme
reduction

| | Retention time | ee value [%] |
|---|---|---|
| Substrate (retention time) | of the product | of the product |
| Acetophenone (16.92 min) | 20.82 min | >99% |
| 4-Chloroacetophenone (21.84 min) | 25.74 min | >99% |
| 2-Oxobutyric acid ethyl ester (10.39 | 13.91 min | >99% |
| min) | | |
| 2-Hexanone | 21.77 min | >99% |
| 2-Heptanone | 14.22 min | >99% |

What is claimed is:

1. A modified enzyme wherein at least one acidic amino acid is replaced with at least one neutral amino acid and wherein the basic amino acids at the coenzyme binding site of said enzyme are not replaced; wherein the modified enzyme exhibits increased NAD(H) affinity compared to an unmodified enzyme.

2. The modified enzyme of claim 1, which is a dehydrogenase enzyme.

3. The modified enzyme of claim 2, which is an alcohol dehydrogenase enzyme.

4. The modified enzyme of claim 2, which is a rec-(R)-alcohol dehydrogenase enzyme.

5. The modified enzyme of claim 4, which is a L. brevis or L. kefir rec-(R)-alcohol dehydrogenase enzyme.

6. The modified enzyme of claim 1, which comprises the amino acid sequence of SEQ ID NO:2.

7. The modified enzyme of claim 5, which is a L. brevis rec-(R)-alcohol dehydrogenase and which comprises a Glycine to Aspartic Acid amino acid change at amino acid 38.

8. An isolated polynucleotide which encodes the modified enzyme of claim 1.

9. The isolated polynucleotide of claim 8, which comprises the nucleotide sequence of SEQ ID NO: 1.

10. A plasmid vector comprising the isolated polynucleotide of claim 8.

11. A host cell comprising the isolated polynucleotide of claim 8.

12. A method of modifying an enzyme comprising: replacing at least one neutral amino acid in said enzyme with at least one acidic amino acid, wherein the basic amino acids at the coenzyme binding site of said enzyme are not replaced; and wherein said modified enzyme exhibits increased NAD(H) affinity compared to an unmodified enzyme.

13. The method of claim 12, wherein said enzyme is a dehydrogenase enzyme.

14. The method of claim 13, wherein said enzyme is an alcohol dehydrogenase enzyme.

15. The method of claim 13, wherein said enzyme is a rec-(R)-alcohol dehydrogenase enzyme.

16. The method of claim 15, wherein said enzyme is a L. brevis or L. kefir rec-(R)-alcohol dehydrogenase enzyme.

17. The method of claim 12, wherein said enzyme comprises the amino acid sequence of SEQ ID NO:2.

18. The method of claim 12, which is a L. brevis rec-(R)-alcohol dehydrogenase and which comprises a Glycine to Aspartic Acid amino acid change at amino acid 38.

19. A method of making the modified enzyme which has improved NAD(H) affinity comprising culturing the cell of claim 8 for a time and under conditions suitable for the expression of the polynucleotide which encodes said enzyme; and collecting the enzyme.

20. The isolated nucleotide sequences of SEQ ID NO:4 and SEQ ID NO:5.

21. Sense and antisense polynucleotides which encode TDRHSDVG.

22. A method of enantioselective reduction of a organic compound comprising reacting said compound with the enzyme of claim 1 and at least one of NAD(H) and NAD+, wherein said organic compound is selected from the group selected from the group consisting of ketones, α-keto esters, β-keto esters, γ-keto esters, and combinations thereof.

23. The method of claim 22, which yields a chiral alcohol.

24. The method of claim 23, wherein said chiral alcohol is an (R)-alcohol.

25. The method of claim 22, wherein said reacting is with NAD(H).

26. The method of claim 22, wherein said enzyme is a dehydrogenase enzyme.

27. The method of claim 22, wherein said enzyme an alcohol dehydrogenase enzyme.

28. The method of claim 22, wherein said enzyme is a rec-(R)-alcohol dehydrogenase enzyme.

29. The method of claim 22, wherein said enzyme is a L. brevis or L. kefir rec-(R)-alcohol dehydrogenase enzyme.

30. The method of claim 22, wherein said enzyme comprises the amino acid sequence of SEQ ID NO:2.

31. The method of claim 22, wherein said enzyme is a L. brevis rec-(R)-alcohol dehydrogenase and which comprises a Glycine to Aspartic Acid amino acid change at amino acid 38.

32. A method of enantioselective oxidation of alcohols comprising reacting an alcohol comprising reacting a alcohol with the enzyme of claim 1 and at least one of NAD(H) and NAD+.

33. The method of claim 32, which yields a chiral alcohol.

34. The method of claim 33, wherein said chiral alcohol is a (R)-alcohol.

35. The method of claim 32, wherein said reacting is with NAD(H).

36. The method of claim 32, wherein said enzyme is a dehydrogenase enzyme.

37. The method of claim 32, wherein said enzyme an alcohol dehydrogenase enzyme.

38. The method of claim 32, wherein said enzyme is a rec-(R)-alcohol dehydrogenase enzyme.

39. The method of claim 32, wherein said enzyme is a L. brevis or L. kefir rec-(R)-alcohol dehydrogenase enzyme.

40. The method of claim 32, wherein said enzyme comprises the amino acid sequence of SEQ ID NO:2.

41. The method of claim 32, wherein said enzyme is a L. brevis rec-(R)-alcohol dehydrogenase and which comprises a Glycine to Aspartic Acid amino acid change at amino acid 38.
